(19) United States
(12) Patent Application Publication
(10) Pub. No.: US 2018/0153745 A1
(43) Pub. Date: Jun. 7, 2018

(54) ORTHOPEDIC SUPPORT APPARATUS AND METHOD OF USE

(71) Applicant: Cast21, Inc., Champaign, IL (US)

(72) Inventors: Jason Troutner, Columbus, OH (US); Ashley Sue Moy, Chicago, IL (US); Andrew Steveson, Champaign, IL (US); Abhliash Seshadri, Chicago, IL (US); Ethan Wisniewski, Champaign, IL (US); Brittany Lung, Park Ridge, IL (US); Kendall Furbee, Hudson, OH (US); Veronica Andrea Hogg-Cornejo, Denver, CO (US)

(73) Assignee: Cast21, Inc., Champaign, IL (US)

(21) Appl. No.: 15/717,069

(22) Filed: Sep. 27, 2017

Related U.S. Application Data
(60) Provisional application No. 62/429,953, filed on Dec. 5, 2016.

(51) Int. Cl. A61F 13/04

(52) U.S. Cl. CPC A61F 13/041; A61F 13/107

(57) ABSTRACT

A support for application to a body area. The support has a network of flexible non-porous tubing interlaced at a plurality of junctions to form a lattice structure. The lattice structure includes apertures to allow for the flow of air and water to the body area. At least one liquid is contained within the network of flexible non-porous tubing and is configured to transform into a solid when acted on by an external stimulus. A padding layer is secured to an internal surface defined on the lattice structure. The padding layer further includes apertures corresponding to the lattice structure apertures such that the flow of air and water to the body area is not impeded by the padding layer.

### FIELD OF THE INVENTION

This invention relates to orthopedic casts, braces, and splints, specifically orthopedic supports comprised of a liquid resin contained in an apertured flexible mold.

### BACKGROUND OF THE INVENTION

Typical casts are made of fiberglass or plaster and have cotton padding. These casts trap water, sweat, bacteria and heat against the patient's skin, leading to discomfort, odor, itchiness, and skin breakdown. These hygiene problems stem from two problems: 1) water and debris trapped under the cast structure, and 2) the inability of the patient to clean the affected area.

Recent innovations have attempted to address the aforementioned problems by using 3D printing to construct waterproof, apertured orthopedic supports. These supports can be fit to the patient by using a 3D scan of the affected limb. The major pitfalls of 3D-printed casts include a high cost, a long time to produce, and the requirement of extra equipment in the form of 3D printers and scanners.

U.S. Pat. No. 4,483,332 (1984) details an orthopedic cast that is constructed by injecting one or more liquids into a network of tubing with at least one inlet, such that one of the liquids hardens into a rigid support structure. Similar to a 3D-printed cast, this method creates a waterproof, breathable cast that can be custom fit to the patient. The potential advantages over a 3D-printed cast are time, cost, and ease of using multiple materials. By using multiple materials, a softer material can provide cushioning while a harder material provides structural support.

### SUMMARY OF THE INVENTION

An improved orthopedic support product and application, which involves a non-porous network of tubing that contains one or more liquids, such that an external stimulus may trigger the transformation of one or more of these liquids into solid form.

In one embodiment of the invention, there is provided a support for application to a body area. The support is defined by a network of flexible non-porous tubing interlaced at a plurality of junctions to form a lattice structure. The lattice structure includes apertures to allow for the flow of air and water to the body area. At least one liquid is contained within the network of flexible non-porous tubing and is configured to transform into a solid when acted on by an external stimulus. In addition, a padding layer is secured to an internal surface defined on the lattice structure. The padding layer further includes apertures corresponding to the lattice structure apertures such that the flow of air and water to the body area is not impeded by the padding layer.

In another embodiment, the at least one liquid is defined to include at least two liquids defined as a resin and a catalyst that transform into a solid when mixed together. The lattice structure may also be configured into two sections and wherein each section holds one of the liquids, and wherein each section is separated by a barrier. The barrier may, in one embodiment, comprise a clip external to the lattice structure and configured in a closed configuration onto the lattice structure at a positioned defined to maintain the two sections of the lattice structure separated and thus the two liquids contained therein separate to prevent mixing. The clip being further configured to have an open configuration to permit the mixing of the two liquids. In another instance, the barrier may comprise a frangible seal internal to the lattice structure. The frangible seal is configured such that applying sufficient pressure to the lattice structure adjacent to one side of the flexible seal breaks said frangible seal, permitting the mixing of the liquids.

In yet another embodiment, the support may include an adhesive layer configured to secure the padding layer to the lattice structure. In yet another embodiment, the support may further include a layer of flexible elastic material secured to a surface of the padding layer that is diametrically opposed to the lattice structure. The layer of flexible elastic material further includes apertures corresponding to the lattice structure apertures such that the flow of air and water to the body area is not impeded by the layer of flexible elastic material.

The support may also include a removable reservoir to receive excess liquid from the at least two liquids.

Numerous other advantages and features of the invention will become readily apparent from the following detailed description of the invention and the embodiments thereof, from the claims, and from the accompanying drawings.

### BRIEF DESCRIPTION OF THE DRAWINGS

A fuller understanding of the foregoing may be had by reference to the accompanying drawings, wherein:

FIG. 1A is a perspective view of an orthopedic support constructed in accordance with one embodiment of the invention and applied to the wrist;

FIG. 1C is a perspective view of an orthopedic support constructed in accordance with one embodiment of the invention;

FIG. 1c is a perspective view of an orthopedic support constructed in accordance with another embodiment of the invention and applied to the wrist with clasps;

FIG. 2A is a cross-sectional view of an orthopedic support construction prior to solidification of the resin, where the flexible sleeve contains two liquid components separated by a divider clip;

FIG. 2B is a perspective view of one type of divider clip in accordance with one embodiment of the invention;

FIG. 2C is a perspective view of the divider clip from FIG. 2B illustrated in a separated configuration;

FIG. 3 shows a reservoir for collecting excess liquid from the main network;

FIG. 4 is an illustration of the liquids separated by a divider clip and contained in an reservoir external to the main tube network;

FIG. 5 is an illustration of two liquids separated by a frangible seal and contained external to the main tube network with a divider clip;

FIG. 6 is a perspective view of a locking mechanism that holds an external reservoir to an inlet on an orthopedic support;

FIG. 7 is a perspective view of a splint constructed in accordance with the invention and applied to the wrist;

FIG. 8A is a cut-away diagram of different layers used to construct an orthopedic support in accordance with one embodiment of the invention;

FIG. 8B is a cut-away diagram of different layers used to construct an orthopedic support in accordance with another embodiment of the invention; and

FIG. 9 is an illustration of an auxiliary, distal component for additional protection of the user and the device against hard surfaces.

### DETAILED DESCRIPTION OF THE EMBODIMENTS

While the invention is susceptible to embodiments in many different forms, there are shown in the drawings and will be described herein, in detail, the preferred embodiments of the present invention. It should be understood, however, that the present disclosure is to be considered an exemplification of the principles of the invention and is not intended to limit the spirit or scope of the invention and/or claims of the embodiments illustrated.

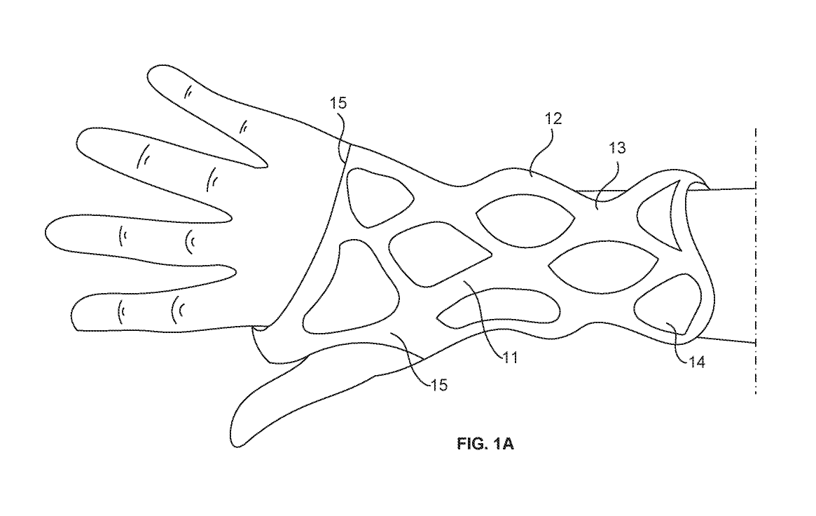

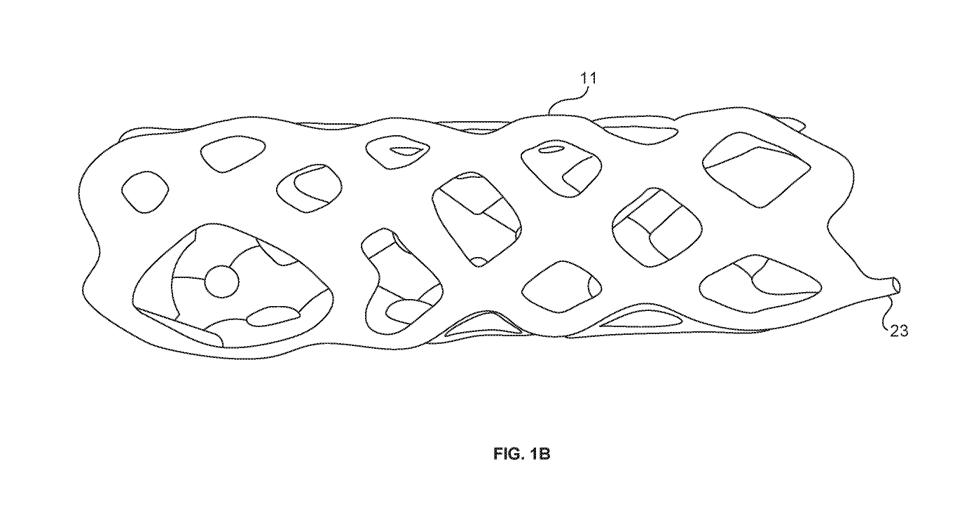

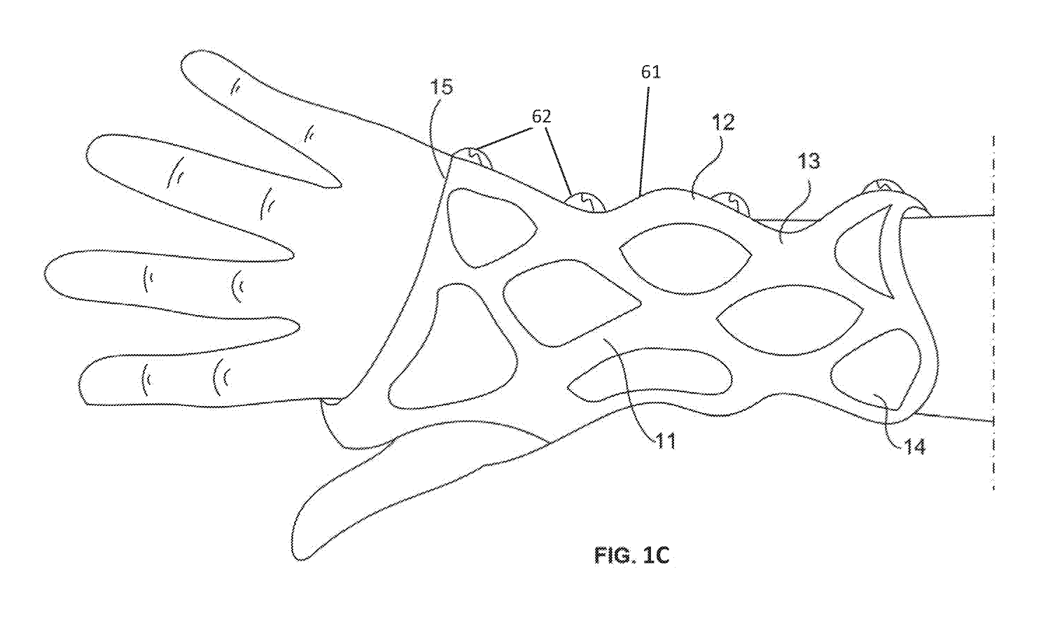

Referring now to FIG. 1A, there is illustrated a perspective view of an orthopedic support 11 constructed in accordance with one or more embodiments of the invention and shown applied to a wrist of a person. The orthopedic support 11 consists initially of a network of flexible non-porous tubing 12. The tubing is interlaced at a plurality of junctions 13 to form a lattice structure. Apertures 14 are included in the lattice structure to allow for expansion of the tubing while it is being applied to a subject, and also allow for the flow of air and water to the subject's skin. Larger apertures 15 may also be included to accommodate body features such as a thumb, fingers, or wrist. As illustrated in the Figures, the network of flexible non-porous tubing can be a single interlaced tube. However, the tubing may also include multiple tubes interconnected. Nonetheless, as defined herein “a network of flexible non-porous tubing” is used to define either embodiments. The tubing also contains one or more liquids, which upon an external stimulus transforms the liquid(s) into a solid. This in turn transforms the liquid inside the initial flexible tubing into a solid and thus preventing the tubing from bending or changing shape (or preventing flexibility) and therefore creating the orthopedic support 11 generally used to refer to a support, cast, brace, or splint, see FIG. 1B.

In another embodiment, FIG. 1C the support 11 is an opened concept that is wrapped around the body area. The open ends of the support would then be secured together, such as with clasps 62 or other means. Multiple clasps may be used to secure the support along its opened edges 61.

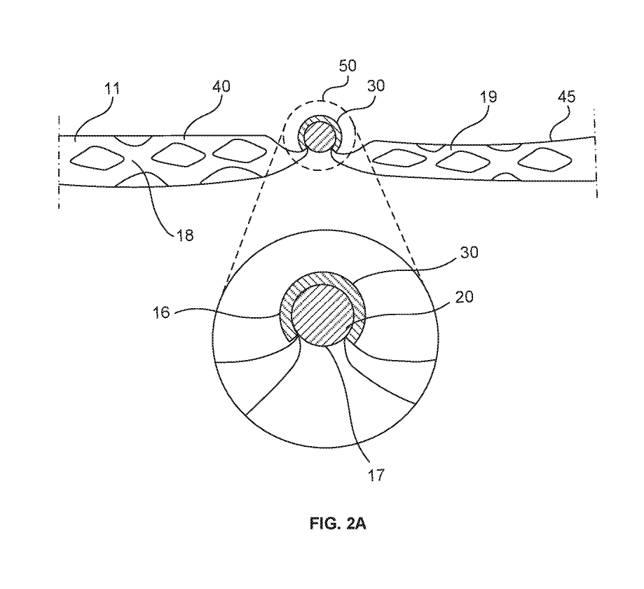

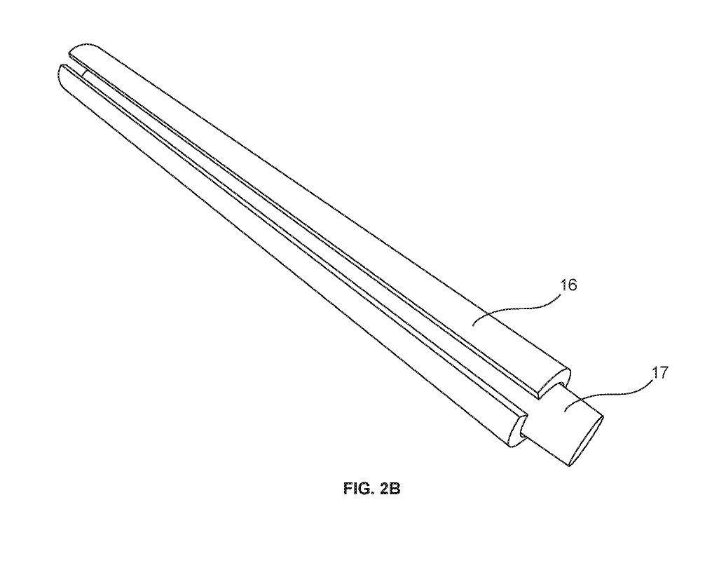

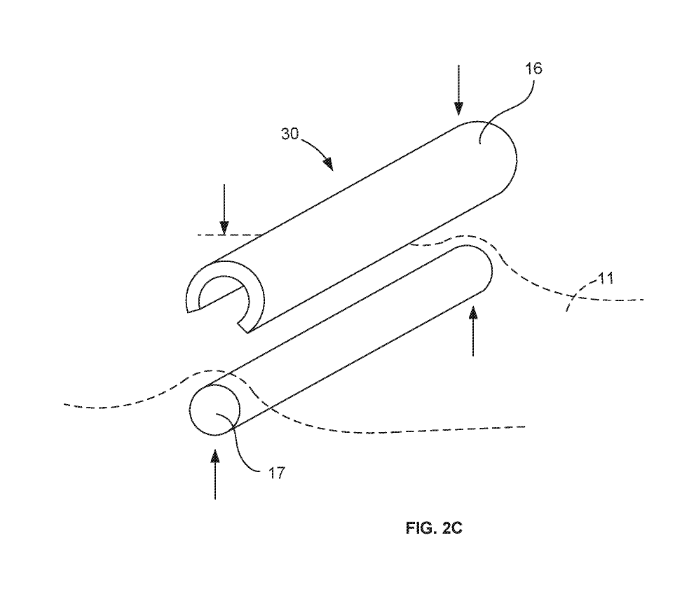

The network of flexible non-porous tubing 12 is preferably made of an elastic material in order to allow bending at the joints and minimize kinking in the tubing. In particular, a thermoplastic elastomer may be used in order to achieve the elastic properties of an elastomer. Manufacturing may be accomplished using laser cutting, film welding, or heating. Referring now to FIGS. 2A through 2C, there is illustrated, in a side view, an orthopedic support 11 in accordance with one embodiment of the invention. The orthopedic support 11 is illustrated prior to solidification of the liquid(s). The orthopedic support 11 includes two support sections 40 and 45 separated by a means of separation 50. In this example, the separation means is a removable divider clip 30. The two support sections 40 and 45 hold two different liquids, a resin 18 and a catalyst 19. In this variation, the external divider clip 30 consists of an outer clip component 16 and an inner clip component 17. The placement of said clip causes a cut-off portion 20 in the tubing, which ensures the separation of the resin and the catalyst. In an alternate manifestation, a frangible adhesive seal on the interior of the tubing may replace the divider clip.

Various other separation means may be utilized, such as a clip that can be activated and deactivated by pressing two components into each other and pulling them apart respectively. In this example, the two components of the clips will have handles on them to facilitate use. In yet another variation of a separation means, there is two levers attached by a hinge that could be activated and deactivated by pressing on the two levers near a fulcrum. A latch on the distal end from the hinge can be used to keep the two levers together.

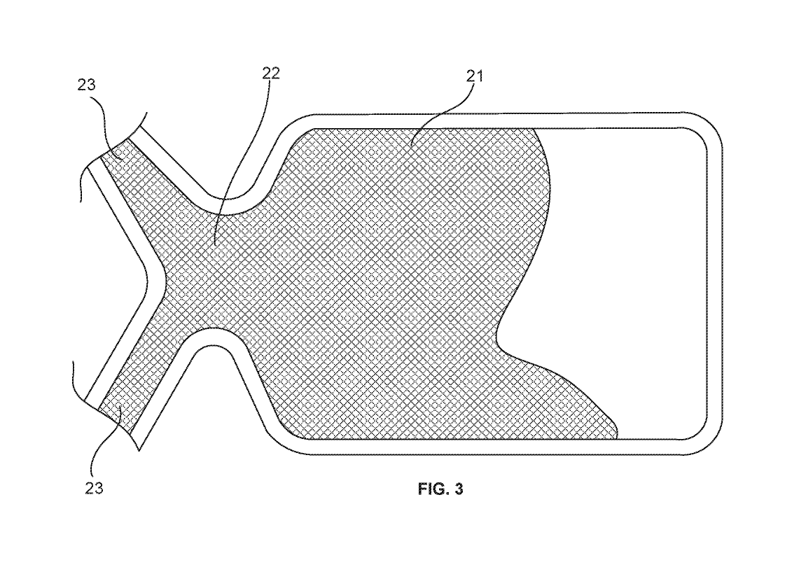

FIG. 3 shows a variation, in which the orthopedic support includes a connected reservoir 21 for excess liquid. This reservoir allows the user to ensure that the orthopedic support remains full, regardless of stretching and deformation that may change the interior volume of the network. The support is attached to the reservoir by one or more support inlets 23 in the support that leads into the inlet 22 of the reservoir. Once the liquid solidifies, the support inlet 23 can be detached from the reservoir. With the liquid solidified, leaking would not be a concern.

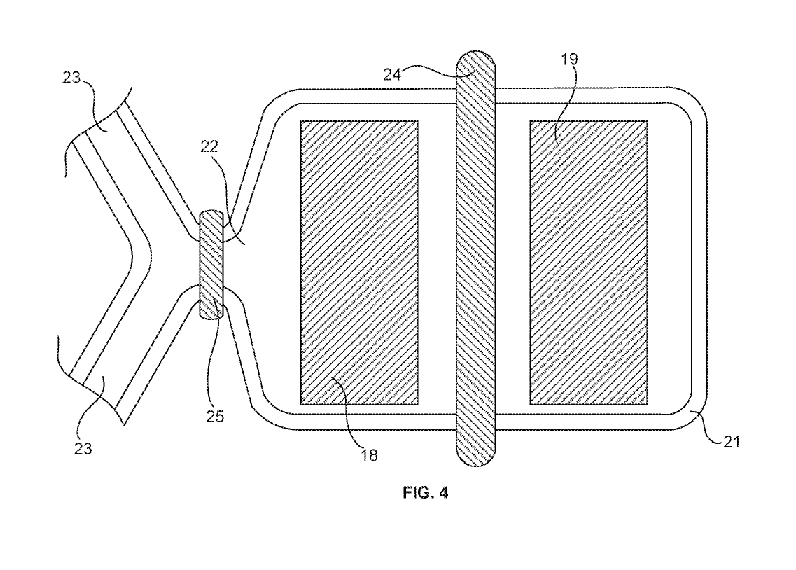

FIG. 4 shows a variation in which the liquid(s) 18 and 19 are initially contained within a large reservoir 21, and the support 11 is initially empty. The reservoir contains a resin 18 and a catalyst 19 separated by a primary divider clip 24. A secondary divider clip 25 placed at the reservoir inlet 22 separates the liquids from the main inlet 23 into the support 11. The user initiates the solidification reaction by removing the primary divider clip 24, then releases the mixed resin into the support by removing the secondary divider clip 25. This variation allows for improved mixing of the resin and catalyst to ensure a uniform reaction.

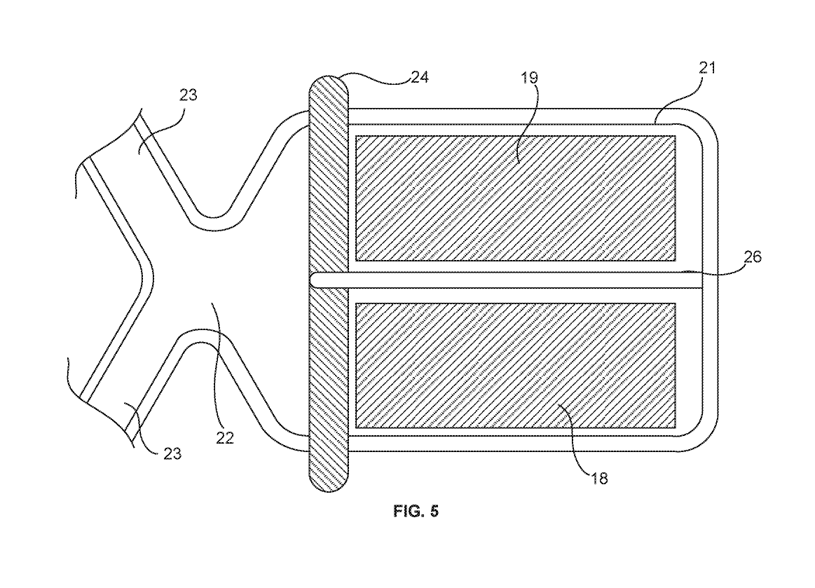

FIG. 5 shows another variation in which the liquid(s) are initially contained within a large reservoir 21, and also with the support 11 initially empty. The reservoir 21 contains a resin 18 and a catalyst 19 separated by a divider seal 26. The resin and catalyst are both contained by a single primary divider clip 24. The user can break the frangible divider seal 26 initiating the solidification reaction and then releases the liquid the reservoir inlet 22 and into the inlets 23 by removing the primary divider clip. This variation allows for an improved ease of use by means of reducing the number of clips to be removed.

In an additional variation, the main divider clip 24 may be replaced by an additional frangible divider seal to reduce the number of clips needed to be removed. This divider in place of the main divider clip can be broken after the divider seal 26 is removed to allow resin to flow to the main network through inlet 22.

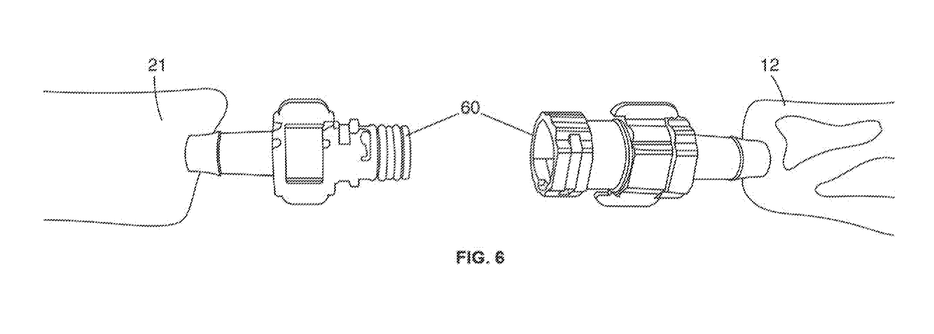

FIG. 6 shows a preferred variation of attachment of the external reservoir to the support 11. It includes a connector 60 that has a series of valves that allow for one-way flow of liquid from an external reservoir to the support. The most preferred variation will allow for this flow of liquid if and only if all of the valve components are connected. This variation is easier for the user to handle the main network separately from the liquids. The quick coupling system uses a shut-off valve design such that only flow occurs when both male and female components are engaged and locked together. A luer lock ensures an easy and secure connection between male and female components during liquid transfer, while a simple Y turn in the opposite direction allows for a quick disengagement of the system once all the liquid from the burst pouch has been dispensed. The internal valve itself is made of Acetal and a surrounding stainless steel 316 spring. When the male and female parts are not engaged the Acetal-spring mechanism acts as a stopper to prevent any leaking of liquid. The ¼″ barbed spout allows for rapid dispensing of the resin into the case sleeve without the need to apply excessive pressure or leaking. Both valve components contain double O-ring and ergonomic flanged handles for an easy press-fit and lock.

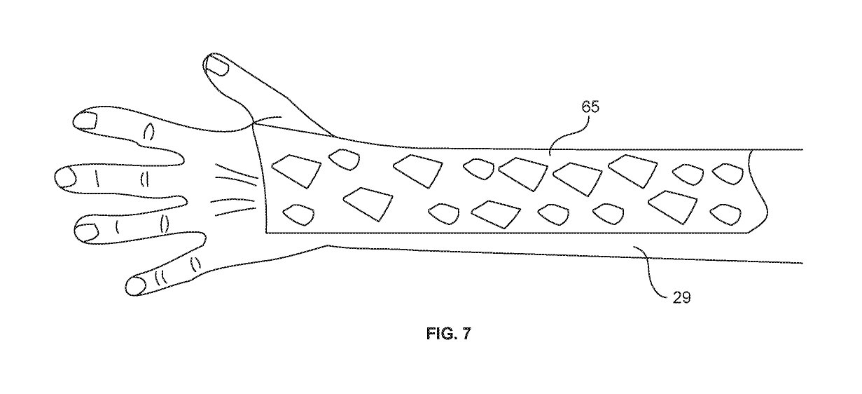

FIG. 7 is a perspective view of a splint 65 constructed in accordance with the invention and applied to the wrist. This variation is functionally similar to the support 11; however, this model is non circumferential without the product touching itself in anyway, leaving a visible, substantial gap 29 or overlay of the material. This variation is preferred for injuries that result in additional swelling or for patients who wish to remove and reuse the product. An additional variation is circumferential but has latches or fasteners in 29 along edge 61 to hold the product as one, cohesive piece.

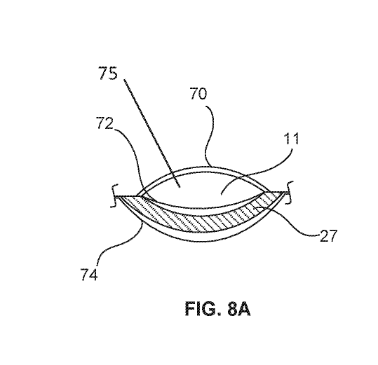

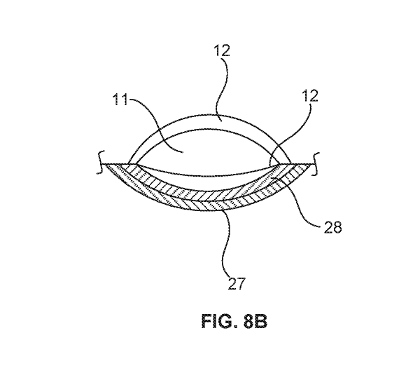

FIG. 8A is a cross section of a portion of the network of the flexible non-porous tubing 12 defined as an upper layer 70 and a lower layer 72 formed into the tubing 12 and filled with the liquid 75 (formed from the combination of resin and catalyst) and which solidifies to create the support 11. As further illustrated, the support 11 may also include an external layer 74 that is defined to come into direct contact with the user and further includes a padding layer 27 positioned between the lower layer 72 and the external layer 74. In another embodiment, illustrated in FIG. 8b, the padding 27 can be secured to an outside surface of one of the layers of the tubing 12 by an adhesive layer 28. In these two embodiments either the padding 27 or an external layer 74 can be positioned against the subject or subject's skin.

These can be incorporated also through cut and welding or melting or by an adhesive or by injection of a liquid or composite that may or may not turn to a solid or semi solid when activated. This variation is preferred if the liquid components in the main network contain liquids that release heat.

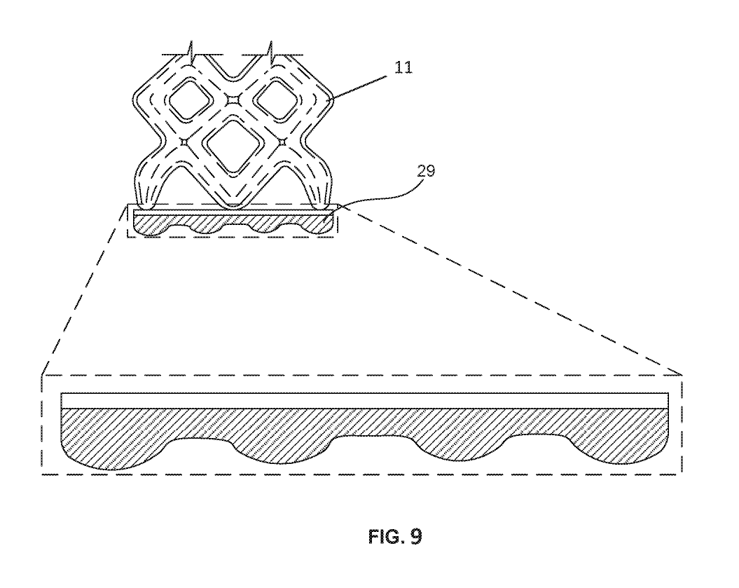

FIG. 9 shows an auxiliary, distal component 29 for additional protection of the user and the device against hard surfaces. A variation of the orthotic, when used to support the lower limb, includes a rigid, rugged distal component 29 to facilitate walking. This component may or may not include substantial apertures. In a preferable variation, this distal component is a laminar composite comprising a rigid layer, such as fiberglass, and a wear-resistant sole material, such as rubber or marine leather. This distal component may either be permanently attached to the support 11 or removably attached by way of one or more buckles or tension or compression or friction.

A variation can include one or more additional seals in the material to prevent liquid flow into targeted areas. This will allow for product removal by shears.

Any of the aforementioned variations can be applied to the supported object, namely a limb or joint, by being placed on the area or pulled over the limb to the desired location.

In a preferable variation, the individual tube segments are initially curved. In this variation, the straightening out of the individual segments allows for further stretching of the structure to fit to the desired shape.

Manufacturing Process

In an ideal variation, the network is created by extruding a thermoplastic elastomer into sheets. These sheets are then cut and welded or sealed with different techniques not limited to RF waves, heat, laser, and friction welding into desired shapes.

A padding layer can be an applied to the support between or on top of any of the layers. In one embodiment, the padding may be a thermoformable foam. Alternative options are thermoresistive padding layers adhered to the top of the sheets or an injectable thermoresistive material or composite such as silicone gel, air, or other liquid(s) that is injected between the extruded sheets of the support after the cut and weld. Additional welds can be made to make the product circumferential or to prevent liquid flow into certain areas. This network, along with any padding layers, is pulled on over an affected area such as a limb or joint or laid on top of it.

Divider clips can be milled or 3D printed from plastic or manufactured by injection molding.

External reservoirs can be pouches with specific frangible seals, wherein two materials are layered on top of each other and bonded together on the perimeter of the pouch. Where the pouch seal needs to be broken, a third material is placed between the original two, and the three materials are bonded together. This seal of all three materials is weaker than the material bond to itself; when pressure is added to the reservoir, this seal will break first.

A series of valves can be produced by but not limited to injection molding and 3D printing can be adhered or welded or held in tension, compression, or with friction to the external reservoir and the main network to hold the two piece together when the valves are engaged. The most ideal variation will only allow one-way flow of liquid if and only if the all of the valves are connected.

Operation

For operation of the variation where one or more liquids have been filled into support 11, the divider clips will be removed to start the solidification process. This occurs before the support is laid on or pulled over an affected area. This variation is ideal for acute care of injuries that will require further medical intervention or for injuries that will result in considerable swelling. This variation can be removed forcibly by hand or with shears.

For operation of the variation where one or more liquids have been filled into an external reservoir, the support is first pulled over or laid on the affected area, namely a joint or limb. Then, the divider clips are removed or the frangible seals are broken to start the reaction and allow flow of the one or more liquids into the network. In the variation which includes a valve connecting system, the valves need to be connected prior to any fluid flow. After the support is filled and or the liquid(s) have solidified, the external reservoirs can be discarded. This variation is ideally removed with a cast saw.

The liquid-filled network of flexible non-porous tubing for any variation can be manipulated around and on the affected area to set bones and adjust joints until solidification. Any distal components are ideally applied post-solidification. An additional variation would involve straps that could attach to clothing or go over the hip or shoulder to hold the device onto a lower limb. In a second variation, the user applies pressure to one side of the network, causing one or more frangible seals to break, and allowing a resin and a catalyst to mix.

After the solidification reaction has been initiated, the user slides the network of tubes onto a limb to be supported. The network of tubes is able to expand to conform to the shape of said limb. Once the solidification reaction has completed, the network becomes a solid support structure.

Alternately, the user may apply the network to the limb prior to activating the solidification reaction. In addition to the aforementioned methods of activating the solidification reaction, this method also allows for solidification by applying a predetermined wavelength of light or temperature to a network containing a photopolymer which will solidify upon exposure to said wavelength of light or heat.

The user may modify the shape of the support by pinching off unwanted segments while the solidification process is occurring. Once the solidification process is complete, the pinched-off segments may be easily cut off.

From the foregoing and as mentioned above, it will be observed that numerous variations and modifications may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

1. A support for application to a body area, the support comprising:
a network of flexible non-porous tubing interlaced at a plurality of junctions to form a lattice structure, and wherein the lattice structure includes apertures to allow for the flow of air and water to the body area;
at least one liquid contained within the network of flexible non-porous tubing, wherein the at least one liquid is configured to transform into a solid when acted on by an external stimulus, and wherein the at least one liquid is configured to release heat when transformed into the solid or the external stimulus uses heat to transform the at least one liquid into the solid; and
a padding layer secured to an internal surface defined on the lattice structure, and wherein the padding layer further includes apertures corresponding to the lattice structure apertures such that the flow of air and water to the body area is not impeded by the padding layer and wherein the padding layer is made of a thermo-resistant material configured to dissipate heat caused during the transformation of the at least one liquid into a solid.

2. The support according to claim 1, wherein said at least one liquid is defined to include at least two liquids defined as a resin and a catalyst that transform into a solid when mixed together.

3. The support of claim 2, wherein the lattice structure is configured into two sections and wherein each section holds one of the liquids, of said at least two liquids, and wherein each section is separated by a barrier configured into the lattice structure.

4. The support according to claim 3, wherein the barrier comprises a clip external to the lattice structure and configured in a closed configuration removably closed onto the lattice structure at a positioned defined to maintain the two sections of the lattice structure separated and thus the two liquids contained therein separate to prevent mixing, and the clip further configured to have an open configuration to permit the mixing of the two liquids.

5. The support according to claim 3, wherein the barrier comprises stacked layers of material internal to the lattice structure and bonded to form a frangible seal internal to the lattice structure, and wherein the frangible seal is configured to be weaker than the lattice structure such that applying sufficient pressure to the lattice structure adjacent to one side of the frangible seal breaks said frangible seal, permitting the mixing of the liquids.

6. The support according to claim 1 further comprising an adhesive layer configured to secure the padding layer to the lattice structure.

7. The support according to claim 1 further comprising a layer of flexible elastic material secured to a surface of the padding layer that is diametrically opposed to the lattice structure, and wherein the layer of flexible elastic material further includes apertures corresponding to the lattice structure apertures such that the flow of air and water to the body area is not impeded by the layer of flexible elastic material.

8. The support according to claim 1, wherein the lattice structure includes a removable reservoir, and wherein the removable reservoir contains excess liquid of the at least one liquid to ensure the lattice structure remains full during the transformation of the at least one liquid to a solid.

9. The support according to claim 1, wherein the lattice structure includes a rigid rugged distal component to facilitate walking.

10. The support according to claim 1, wherein the liquid is a photopolymer.

11. The support according to claim 1, wherein the lattice structure includes one or more clasps, and the one or more clasps being configured to secure the lattice structure around the body area when closed and further configured to allow removal of the lattice structure from the body area when opened.

12. The support according to claim 1, wherein the lattice structure is in the shape of the appendage to which it is intended to be applied.

13. The support according to claim 1, wherein the lattice structure is configured to be wrapped around the appendage and connected with clasps.

14. A system to create a support for application to a body area, the support comprising:
a network of flexible non-porous tubing interlaced at a plurality of junctions to form a lattice structure, and wherein the lattice structure includes apertures to allow for the flow of air and water around the network of flexible non-porous tubing to the body area;
a padding layer secured to an internal surface defined on the lattice structure, and wherein the padding layer further includes apertures corresponding to the lattice structure apertures such that the flow of air and water to the body area is not impeding by the padding layer;
an inlet attached to the lattice structure to permit the flow of one or more liquids into the network of flexible non-porous tubing; and
an external reservoir containing one or more liquids and a barrier between the inlet and the one or more liquids such that when the reservoir is attached to the inlet the one or more liquids are added to the lattice structure manually and wherein the one or more liquids are configured to transform into a solid when acted on by an external stimulus, and wherein the one or more liquids are selected from a liquid configured to release heat when transformed into the solid or the external stimulus uses heat to transform the one or more liquids into the solid and wherein the padding layer is made of a thermo-resistant material configured to dissipate heat caused during the transformation of the at least one liquid into a solid.

15. The system of claim 14, wherein the reservoir contains two sections separated by a frangible seal, and the two sections separately contain a resin and a catalyst that transform into a solid when mixed together and the frangible seal is configured to rupture when sufficient pressure to applied to one side of the frangible seal.

16. The system of claim 14 further comprising a valve positioned between inlet and reservoir to control the flow of the one or more liquids into the lattice structure.

17. The system of claim 14 further comprising an adhesive layer configured to secure the padding layer to the lattice structure.

18. The system of claim 14 further comprising a layer of flexible elastic material secured to a surface of the padding layer that is diametrically opposed to the lattice structure, and wherein the layer of flexible elastic material further includes apertures corresponding to the lattice structure apertures such that the flow of air and water to the body area is not impeded by the layer of flexible elastic material.

19. The system of claim 14, wherein the reservoir is removably attached to the inlet.

20. The system of claim 14, wherein the barrier is a removable clasp.
